(19) United States
(12) Patent Application Publication
(10) Pub. No.: US 2010/0021427 A1
(43) Pub. Date: Jan. 28, 2010

(54) Use of Antiviral Peptides For Treatment of Infections Caused by Drug-Resistant HIV

(75) Inventors: Xin Chen, Tianjin (CN); Linqi Zhang, Edison, NJ (US); Shibo Jiang, New York City, NY (US)

Correspondence Address: Milstein Zhang & Wu LLC, 49 Lexington Street, Suite 6, Newton, MA, 02465-1062, US

(73) Assignee: Tianjin FusoGen Pharmaceuticals, Inc., Tianjin (CN)

(21) Appl. No.: 12/474,702

(22) Filed: May 29, 2009

Related U.S. Application Data
(60) Provisional application No. 61/057,102, filed on May 29, 2008.

(51) Int. Cl. A61K 38/16; A61P 31/18; A61K 38/21

(52) U.S. Cl. 424/85.5; 514/12; 424/85.6; 424/85.7

(57) ABSTRACT

The present invention provides methods of treating drug-resistant HIV infections especially of HIV strains that are resistant to infusion inhibitors, such as T20.

### CROSS-REFERENCE TO RELATED APPLICATIONS

This application claims priority to and the benefit of U.S. provisional patent application Ser. No. 61/057,102 filed on May 29, 2008, the entire content of which application is incorporated herein by reference.

### FIELD OF THE INVENTION

This invention relates to use of antiviral peptides for the treatment of viral infection, especially to method for treatment of viral infection caused by drug-resistant HIV.

### BACKGROUND OF THE INVENTION

### 1. AIDS Epidemic

Infection with Human Immunodeficiency Virus (HIV), a pathogenic retrovirus, can cause Acquired Immunodeficiency Syndrome (AIDS) (Barre-Sinossi, F. et al. SCIENCE. 1983; 220: 868-870). Although macrophage, neuron and other cells can be infected by HIV (Maddon et al. CELL. 1986; 47:333-348), the CD4+ lymphocytes are the major target cells for HIV (Dalgleish, A. et al. NATURE. 1984; 312:767-768), because HIV has strong affinity to the CD4 molecules on the surfaces of CD4+ cells. HIV infection in a human body destroys so many CD4+ lymphocytes that the body begins to lose its immune function, therefore an AIDS patient is highly vulnerable to various infections, neuronal dysfunction, tumors, and so on. Without effective treatment, these infections can quickly become lethal (Levy, J. A., ed., ACUTE HIV INFECTION AND SUSCEPTIBLE CELLS, published in U.S.A, 2000, p 63-78).

With its severe symptoms and high mortality rate, the epidemic contagion of AIDS has become one of the leading causes of death that is threatening human health. So far in the entire world, people infected by HIV have accumulated to a total of 33.2 million. In 2007 alone, 2.1 million died from AIDS and 2.5 million people became infected by HIV. Every day, over 6800 persons become infected with HIV and over 5700 persons die from AIDS. The United States of America is one of the countries with the largest number of HIV infections in the world. (WHO Report 2007, UNAIDS and WHO).

Currently, at least two types of HIV have been identified: HIV-I (Gallo, R. et.al. SCIENCE. 1984; 224: 500-503) and HIV-2 (Clavel, F. et al. SCIENCE. 1986; 223:343-346). Each of them has high genetic heterogeneity. For HIV-1 strains alone, they can be classified into three groups: the “major” group M, the “outlier” group O and the “new” group N. Group M can be subdivided into at least 9 subtypes, designated A, B, C, D, F, G, H, J and K, together with circulating intersubtype recombinants designated CRF01_AE (formerly called subtype E) to CRF19_cpx (formerly called subtype 1). (Robertson et al. SCIENCE 2000; 288 (5463): 55). On a global scale, the most prevalent HIV-1 genetic forms are subtypes A, B, C, D, and two of the CRF, CRF01-AE and CRF02_AG, respectively. Subtype A distributes mainly in East Africa, subtype B is widespread globally, dominating epidemics in the Americas, Europe, and Australia, subtype C accounts for more than 50% of all infections worldwide, concentrated in Southern and East Africa, and in India. Subtype D circulates in East Africa. CRF01_AE and subtype B is the major strain throughout Southeast Asia and CRF02_AG dominates West Africa. (McCutchan F. E. J. MED. VIROL. 2006; 78: S7-S12).

### 2. Human Immunodeficiency Virus, HIV

The reproduction cycle of HIV has several important steps. First, the envelope glycoprotein gp 120 of the virus attaches to the host cell membrane through its specific binding with CD4 molecule located on the surface of T4 lymphocyte. With the assistance of a series of chemokine co-receptors, the viral envelope fuses with the host cell membrane (Berger, E. A., et al. ANN. RE. IMMUNOL. 1999; 17: 657-700). After the fusion process, the HIV virion packed in nucleocapsid enters into the host cell with its capsid shucked off and viral nucleic acid exposed. The viral reverse transcriptase catalyzes the transcription of HIV single-stranded RNA into single-stranded DNA, which is then transformed to double-stranded DNA by the catalysis of cellular polymerase. The double-stranded DNA can either exist freely in cytoplasm or be integrated as provirus into host chromosome's DNA by the catalysis of viral integrase, thus engendering HIV latent infection (Roe, T. et al. J. VIROL. 1997; 71(2): 1334-40). Provirus, which will not be excised from the host chromosome, is very stable and reproduces itself with the replication of host chromosome. After the HIV mRNA is translated into a large polyprotein, the viral proteases cut and process the polyprotein to form mature viral structural proteins (Xiang, Y. & Leis, J. J. VIROL. 1997; 71(3): 2083-91). These structural proteins, together with HIV nucleic acids, are firially assembled into new virus granules and released outside the cell through budding (Kiss-Lazozlo, Hohn, T. TRENDS IN MICROBIOLOGY. 1996; 4(12): 480-5).

In sum, the critical stages of HIV replication are: 1) virus attachment and entry into host cell through a fusion process; 2) reverse transcription and integration; 3) protein translation and processing; 4) virus assembly and release.

3. Fusion Inhibitors that Block Viral Entry into Cells

Membrane fusion is a critical step for the virus to attack and penetrate the host cells (Weissenhorn, W., et al. NATURE. 1997; 387:426-430). The fusion process is controlled by the glycoproteins on HIV envelope. The precursor of the glycoproteins is gp160 that has polysaccharide groups. During the viral reproduction period, gp160 is hydrolyzed by host protease into two subunits: gp120 anchoring outside the envelope and transmembrane protein gp41. After the hydrolyzation, gp120 and gp41 are still linked by a non-covalent bond and polymerized as trimers outside the viral granule. The transmembrane protein gp41, whose ectodomain with a highly helical structure, has a highly efficient origination mechanism for membrane fusion, and is known as the pivotal molecule for opening the gate of cells for its direct participation in the fusion process (Ferrer, M., et al. NAT. STRUCT. BIOL. 1999; 6(10): 953-60; Zhou, G., et al. BIOORG. MED. CHEM. 2000; 8(9): 2219-27).

Crystal diffraction analyses have shown that when fusion takes place between the HIV virus and the cell, the core of gp41 consists of six helical bundles (6-HB) wherein the N-terminal and C-terminal helices are collocated as three hairpins which fix the HIV envelope to the cellular membrane. The gp41 trimer can form a fusion pore that facilitates the viral intrusion into the host cell (Chan, D. C., et al. CELL. 1997; 89: 263-273). Gp4l exists in an unstable natural non-fusion conformation on the surface of the free virus particle fresh sprouting from infected cells. At first, the N-terminal helices are wrapped inside the C-tenninal helices so that the N-terminal fusion area is hidden, then after gp120 on the viral surface combines with the CD4 receptor and chemokine co-receptors on the cell membrane, a receptor-activated conformational change of gp41 occurs wherein its N-terminal extends beyond the viral surface into the host cellular membrane. At this time, gp41 is transformed from an unstable natural non-fusion conformation into a pre-hairpin intermediate conformation. When the C-terminal and N-terminal of gp41 peptides bind together, the hydrophobic N-terminal core of the trimer structure is exposed, and the pre-hairpin intermediate is transformed into a more energy-stabilized hairpin conformation. By this time the viral envelope has fused with the cellular membrane (Jones, P. L., et al. J. BIOL. CHEM. 1998; 273:404). Using proteolytic dissection strategies, Lu et al. (Lu, M., et al. NAT. STRUCT. BIOL. 1995; 2: 10751082; Lu, M., et al. J. BIOMOL. STRUCT. DYN. 1997; 15: 465-471) isolated several pairs of protease-resistant N- and C-peptides from gp41 including N36 and C34, which was later found to form the stable fusogenic core. C34 and N36 have been used as laboratory tools to test the concept and mechanism of anti-fusion treatement (Liu, S. et al. Curr. Pharm. Des., 2007,13(2):143; Liu, S. et al. J. Biol. Chem.2005; 280(12): 11259-11273).

### 4. Development of Drug Resistance

Great efforts have been dedicated to devising effective anti-HIV medicines for many years. After the introduction of highly active antiretroviral therapy (HAART), combinatory therapy using different classes of antiretroviral drugs that act at different stages of the HIV life cycle, the morbidity and mortality of HIV/AIDS decreased dramatically (Palella F. J., et al., N. ENGL. J. MED., 1998; 338: 853-860). However, along with the global utilization of HAART, increasing amount of drug resistant HIV compromised the therapy effect, and a minority of patients might face the exhaustion of available treatment (Sabin C A, et al., B.M.J., 2005, 330: 695-699).

It has been reported that in cases where antiviral therapy failed after one-year treatment, about 50% of them were due to drug resistance (Montaner J. S., et al., LANCET, 1998; 352: 1919-1922). Besides, resistant HIV transmission caused by resistant gene kept rising. In Europe, 10.4% treatment-naive patient carries HIV strains with drug resistant gene mutation (Wensing AMJ, et al., J INFEC DIS, 2005; 192: 958-966). HIV resistant mutations updated by International AIDS Society-USA are 42 in 1994, 236 in 2000, and more than 300 in 2005.

Reverse transcriptase inhibitor, including AZT, ddI, ddC, 3TC, and d4T, etc., would induce drug resistance, sooner or later, the viruses become less sensitive to the drug, and the effective inhibition concentration of the drug rise by several-fold or even ten-fold (Vella, S. and Floridia, M. INTERNATIONAL AIDS SOCIETY USA. 1996; 4(3):15).

This drug-resistance is associated with high mutation rate of HIV. In a human body, a single HIV virus could produce 10⁸-10¹⁰ new virus granules every day, while the mutation rate is 3×10⁵ per replication cycle. Many mis-sense mutations, affecting the expression of amino acids, may happen in the regulatory genes as well as in the envelope proteins. In some HIV strains, the mutation rate could be as high as 40% in the amino acid sequences of certain genes (Myers, G. and Montaner, J. G. The Retroviridae vol. 1, Plenum Press, New York 1992; 51-105). By suppressing drug-sensitive strains using reverse transcriptase inhibitors, we unwittingly facilitate the proliferation of resistant strains that exist before and after the mutations. As a result, drug-resistance develops. [00014] More than 50 reverse transcriptase mutations are associated with resistance against nucleoside reverse transcriptase inhibitor (NRTI). The NRTI resistance mutations include M184V, thymidine analog mutations (TAMs), mutations selected by regimens lacking thymidine analogs (Non-TAMs), and multi-nucleoside resistance mutations (Multi-NRTI mutations) and many recently described non-polymorphic accessory mutations. There are two biochemical mechanisms of NRTI resistance: enhanced discrimination against and decreased incorporation of NRTI in favor of authentic nucleosides, and enhanced removal of incorporated NRTI by promoting a phosphorolytic reaction that leads to primer unblocking. Altogether, M184V, non-thymidine-analog-associated mutations such as K65R and L74V, and the multi-nucleoside resistance mutation Q151 M act by decreasing NRTI incorporation. Thymidine analog mutations, the T69 insertions associated with multi-nucleoside resistance, and many of the accessory mutations facilitate primer unblocking. (AIDS REV. 2008; 10 (2): 67-84).

M184V is the most commonly occurring NRTI resistance mutation. In vitro, it causes high-level resistance to lamivudine (3TC) and emtricitabine (FTC), low-level resistance to didanosine (ddI) and abacavir (ABC), and increased susceptibility to zidovudine (ZDV), Stavudine (d4T), and tenofovir (TDF). (AIDS REV. 2008; 10(2): 67-84).

Thymidine analog mutations (TAMs) are selected by the thymidine analogs ZDV and d4T. TAMs decrease susceptibility to ZDV and d4T, and to a lesser extent to ABC, ddI, and TDF. The most common drug-resistant amino acid mutations are M41L, D67N, K70R, L210W, T215Y/F and K219QE. The presence of each mutation of M41L, L20W, and T215Y can cause higher levels of phenotypic and clinical resistance to thymidine analogs and cross-resistance to ABC, ddI, and TDF. (AIDS REV. 2008; 10(2): 67-84).

The most common mutations in patients developing virologic failure while receiving a non-thymidine analog containing NRTI backbone (Non-TAMs) include M184V alone or M184V in combination with K65R or L74V. K65R causes intermediate resistance to TDF, ABC, ddI, 3TC, and FTC, low-level resistance to d4T, and increased susceptibility to ZDV. Mutations of M184V plus K65R have been reported primarily in patients receiving the NRTI backbone TDF/3TC and less commonly with ABC/3TC or TDF/ FTC. L74V causes intermediate resistance to ddI and ABC, and a slight increase in susceptibility to ZDV and TDF. M184V plus L74V occurs primarily in persons receiving ABC/3TC or ddI/3TC/FTC backbones. L741 has similar phenotypic properties to L74V, but is found primarily in viruses with multiple TAM. Mutations in bold are associated with higher levels of phenotypic resistance or clinical evidence for reduced virologic response. Other Non-TAMs mutations include K65N, K70E/G, L741, V75T/M, Y115F (AIDS REV. 2008; 10(2):67-84).

Amino acid insertions at codon 69 generally occur in the presence of multiple TAM, and in this setting are associated with intermediate resistance to 3TC and FTC and high-level resistance to each of the remaining NRTI. Q151 M is a 2-bp mutation (CAG→ATG) that is usually accompanied by two or more of the following mutations: A62V, V751, F77L, and F116Y. The Q151M complex causes high-level resistance to ZDV, d4T, ddI, and ABC, and intermediate resistance to TDF, 3TC, and FTC (AIDS REV. 2008; 10(2): 67-84).

More than 40 reverse transcriptase mutations are associated with resistance against non-nucleoside reverse transcriptase inhibitor (NNRTI). These mutations include major primary and secondary mutations, non-polymorphic minor mutations, and polymorphic accessory mutations. The NNRTI inhibit HIV-1 RT allosterically by binding to a hydrophobic pocket close to but not contiguous with the RT active site. Nearly all NNRTI resistance mutations are within the NNRT1 binding pocket or adjacent to residues in the pocket. There is a low genetic barrier to NNRTI resistance, with only one or two mutations required for high-level resistance. High levels of clinical cross-resistance exist among the NNRTI because many of the NNRTI resistance mutations reduce susceptibility to multiple NNRTI and because the low genetic barrier to resistance allows a single NNRTI to select for multiple NNRTI resistance mutations in different viruses, even if only a single mutation is detected by standard population-based sequencing.

Each of the primary NNRTI resistance mutations—K103N/S, V106A/M, Y181C/I/V, Y188L/C/H, and G190A/S/E—causes high-level resistance to nevirapine and variable resistance to Efavirenz, ranging from about 2-fold for V106A and Y181C, 6-fold for G190A, 20-fold for K103N, and more than 50-fold for Y188L and G190S.

L1001, K101P, P225H, F227L, M230L, and K238T are secondary mutations that usually occur in combination with one of the primary NNRTI resistance mutations. L1001 and K101P, which occur in combination with K103N, further decrease NVP and EFV susceptibility from 20-fold with K103N alone to more than 100-fold. V179F, F227C, L2341, and L318F are rare mutations that are important since the license of etravirine.

Minor non-polymorphic mutations—A98G, K101 E, V 1081, and V 179D/E are common NNRTI resistance mutations that reduce susceptibility to nevirapine and efavirenz about 2-fold to 5-fold.

Miscellaneous nonnucleoside reverse transcriptase inhibitor resistance mutations, such as K101Q, 1135T/M, V1791, and L2831, reduce susceptibility to nevirapine and efavirenz by about twofold and may act synergistically with primary NNRTI resistance mutations. Other mutations such as L74V, H221Y, K223E/Q, L228H/R, and N3481 are selected primarily by NRTI, yet also cause subtle reductions in NNRTI susceptibility.

Moreover, all the reverse transcriptase inhibitors have specific toxicity related to their doses. The symptoms include spinal cord suppression, vomiting, liver dysfunction, muscle weakness, diseases of peripheral nervous system, and pancreatic inflammation. Many patients have to suspend the treatment due to these intolerable side effects (Fischl, M. A., et al. N. ENGL. J. MED. 1987; 317: 185-91; Lenderking, W. R., et al. N. ENGL. J. MED. 1994; 330: 738-43).

Drug-resistance is also a problem for protease inhibitor. More than 60 mutations are associated with protease inhibitor (PI) resistance including major protease, accessory protease, and protease cleavage site mutations (AIDS REV. 2008; 10(2):67-84). Seventeen largely non-polymorphic positions are of the most clinical significance, including L231, L241, D30N, V321, L33F, M461/L, 147/V/A, G48V/M, 150L/V, F53L, 154V/T/A/L/M, G73S/T, L76V, V82A/T/F/S, 184V/A/C, N88D/S, L90M. Of these 17 positions, 13 mutations have been shown to reduce susceptibility to one or more PI. Accessory protease mutations include the polymorphic mutations L101/V, 113V, K20R/M/I, M36I, D60E, I62V, L63P, A71V/T, V77I, and 193L and the non-polymorphic mutations L10F/R, V111, E34Q, E35G, K43T, K45I, K55R, Q58E, A71I/L, T74P/A/S, V751, N83D, P79A/S, 185V, L89V, T91 S, Q92K and C95F.

Side effects of protease inhibitors include liver dysfunction, gastrointestinal discomfort, kidney stone, numbness around mouth, abnormality of lipid metabolism, and mental disorder (Deeks, et al. JAMA 1997; 277:145-53).

Although fusion inhibitor is a new kind of anti-HIV drug, drug resistant strains have already developed in clinical practice (Xu L. et al. ANTIMICROB AGENTS CHEMOTHER. 2005; 49(3): 1113-1119). The first-discovered fusion inhibitor, T20 or enfuvirtide, is a 36 amino-acid peptide derived from the C-terminal (127-162) of gp41. The structural similarity of T20 to the C-terminal of gp41 makes it capable of competing against the C-peptide of gp41 in binding to the N-terminal fusion domain. More than 15 gp41 mutations are associated with the fusion inhibitor Enfuvirtide. Drug resistant amino acid mutations of T20 mainly develop in the 36-45 amino acid residues of gp41 HR1 to which Enfuvirtide binds, and are primarily responsible for Enfuvirtide resistance. Most commonly observed Enfuvirtide resistance mutations in this region include G36D/E/V/S, 137V, V38E/A/M/G, Q40H, N42T, N43D/K/S, L44M, L45M. A single mutation is generally associated with about 10-fold decreased susceptibility, whereas double mutations can decrease susceptibility by more than 100-fold (AIDS REV. 2008; 10(2):67-84). Besides HR1, the HR2 domain of gp41 is also associated with T20 resistance (Xu L. et al. ANTIMICROB AGENTS CHEMOTHER. 2005; 49(3): 1113-1119).

Due to the drawback of T20 aforementioned, researchers have developed other novel fusion inhibitors with improved properties. A peptide used for treating HIV infection is published in co-owned Chinese Patent CN01130985.7, the entire disclosure of which is incorporated herein by reference. The anti-viral peptide is derived from the amino acid residues 117-151 of the C-terminal of HIV-1 (E subtype) transmembrane protein gp41. Specifically, the amino acid sequence of the peptide is: X-SWETWEREIENYTKQIYKILEESQEQQDRNEKDLLE-Z. It has been reported that the IC₅₀ of the peptide is 20-time lower than T20 in an HIV fusion assay. Pharmaceutical compositions of an antiviral peptide have been published in a co-owned application PCT/CN2007/001849, published as WO 2007/143934 and its entire content is also incorporated herein by reference.

In sum, most of the currently used anti-HIV drugs have their limits, such as high toxicity and/or propensity for inducing drug resistance. Therefore, there is still a huge and urgent need for new anti-HIV drugs with higher efficacy and lower toxicity, and especially for drugs that retain inhibitory activities against drug-resistant HIV strains. In this invention, we discovered the mechanism of action of a series of antiviral peptides, which show strong inhibitory activity against drug resistant HIV, including HIV strains that have been found to be resistant to existing fusion inhibitors.

### SUMMARY OF THE INVENTION

In one aspect, the present invention relates to the use of antiviral peptides that include the amino acid sequence of SWETWEREIENYTRQIYRILEESQEQQDRNERDLLE (SEQ ID NO:1) or a conservatively modified variant for treatment of infections caused by drug resistant HIV strains, especially of infections caused by fusion inhibitor resistant strains, e.g., T20.

In another aspect, the present invention is directed to the use of a conservatively modified variant of SEQ ID NO:1 with the amino acid sequence of X-SWETWEREIENYTRQIYRILEESQEQQDRNERDLLE-Z for treatment of HIV strains that are resistant to at least one existing HIV medication. “X” may represent an amino group, a hydrophobic group, including but not limited to carbobenzoxyl, dansyl, or tert-butyloxycarbonyl; an acetyl group; 9-fluorenylmethoxy-carbonyl (Fmoc) group; a macromolecular carrier group including but not limited to lipid fatty acid conjugates, polyethylene glycol, or carbohydrates. “Z” may represent a carboxyl group; an amido group; a tert-butyloxycarbonyl group; a macromolecular carrier group including but not limited to lipid fatty acid conjugates, polyethylene glycol, or carbohydrates.

In another aspect, the present invention is directed to a method of treating infection caused by drug-resistant HIV comprising administering to the patient a therapeutically effective amount of at least one of the peptides shown above or a mixture of mutiple peptide componenets thereof.

The present invention also provides a method of treating viral infection caused by drug-resistant HIV, including administrating an effective amount of aforementioned antiviral peptides and at least one other therapeutic agent, e.g., another anti-HIV medication. In one embodiment, the additional agent is selected from the group consisting of reverse transcriptase inhibitors, protease inhibitors, integrase inhibitors, glycosidase inhibitors, viral mRNA capping inhibitors, amphotericin B, ester-bond binding molecules with anti-HIV activity, hydroxyurea, α-interferon, β-interferon, γ-interferon, and other anti-HIV agents.

In yet another aspect, the present invention is directed to a method of treating drug-resistant HIV infection in a subject in need thereof, comprising administering to said subject at least a genetic material capable of producing inside the subject a therapeutically effective amount of a peptide comprising the amino acid sequence of SEQ ID NO:1 or a conservatively modified variant thereof.

In one embodiment, the peptide is administered in a manner selected from the group consisting of intramuscular, intravenous, subcutaneous, oral, mucosal, rectal and percutaneous administration.

### BRIEF DESCRIPTION OF THE DRAWINGS

The foregoing, and other features and advantages of the invention, as well as the invention itself, will be more fully understood from the description, drawings and claims that follow. The drawings are not necessarily to scale, emphasis instead generally being placed upon illustrating the principles of the invention.

FIG. 1 is a chart that illustrates the result of a CD spectroscopy analysis of the secondary structures of FS-01, C34, T20 alone and their respective complexes with N36 thereof.

FIG. 2 is a chart illustrating thermostability analysis of the complexes formed by N36/FS-01 or N36/C34. The inset is the first derivative of the curve against temperature, which was used to determine the Tm value.

FIG. 3 is the image of the native-PAGE gel electrophoresis showing the 6-HB formation between FS-01, T20, C34 and N36.

FIG. 4 is a chart showing inhibitory activities of FS-01 on 6-HB formation measured by ELISA.

FIG. 5 is a chart showing the inhibitory effect of FS-01 and T20 on HIV-1IIIB-mediated cell-cell fusion.

FIG. 6 is a chart showing the inhibitory effect of FS-01 and T20 on HIV-1IIIB-mediated infection of MT-2 cells.

FIG. 7 consists of diagrams showing the heat release of FS-01, T20 and C34 when mixed with POPC LUVs. Upper panel shows the titration traces when T20 is mixed with N36. Lower panel shows respective binding affinity of T20, C34 or FS-01 to N36.

FIG. 8 consists of diagrams showing the fluorescence spectroscopy of FS-01, T20 and C34 alone and in mixture with POPC LUVs. Upper panel shows the fluorescence spectra of peptides in blank PBS solutions. Lower panel shows the fluorescence spectra of peptides mixed with POPC LUVs.

### DETAILED DESCRIPTION OF THE INVENTION

According to the aforementioned, the present invention provides use of antiviral peptides to treat viral infection caused by drug-resistant HIV strains, such as those resistant to reverse transcriptase inhibitors, protease inhibitors or fusion inhibitors.

According to the antiviral principle of the aforementioned antiviral peptide, the active part of the peptide is the 36 amino-acid sequence (SEQ ID NO:1). Thus, the peptide can be conservatively modified according to methods well known to one skilled in the art to the extent that the modification doses not destroy α-helices, but can increase the stability, bioavailability or activity of the peptide.

Thus, one aspect of the present invention is directed to the use of a conservatively modified variant of SEQ ID NO:1 with the amino acid sequence of X-SWETWEREIENYTRQIYRILEESQEQQDRNERDLLE-Z for treatment of infection caused by drug-resistant HIV strains, “X” may represent an amino group, a hydrophobic group, including but not limited to carbobenzoxyl, dansyl, or tert-butyloxycarbonyl; an acetyl group; 9-fluorenylmethoxy-carbonyl (Fmoc) group; a macromolecular carrier group including but not limited to lipid fatty acid conjugates, polyethylene glycol, or carbohydrates. “Z” may represent a carboxyl group; an amido group; a tert-butyloxycarbonyl group; a macromolecular carrier group including but not limited to lipid-fatty acid conjugates, polyethylene glycol, or carbohydrates.

In one embodiment, the peptide is: CH3CO-SWETWEREIENYTRQIYRILEESQEQQDRNERDLLE-NH2 (refered to as FS-01 hereinafter).

In additional embodiments, the present invention provides segments of the antiviral peptide (SEQ ID NO:1) that shows activity against drug resistant HIV strains. These truncated antiviral peptides can contain 4-36 amino acids (namely, ranging from a tetrapeptide to a 36-amino-acid peptide).

In addition to the whole sequence of the antiviral peptide (SEQ ID NO:1) and the truncated antiviral peptide, the composition of the present invention can also include peptides comprising SEQ ID NO:1, or peptides with substitution, insertion, and/or deletion of one or more amino acids in the sequence of SEQ ID NO:1. The aforementioned peptide comprising SEQ ID NO:1 can be produced by adding 2-15 amino acids on a terminal of SEQ ID NO:1. The amino acid insertion can be made up of a single amino acid residue or a residue segment of 2-15 amino acids. According to an embodiment of the present invention, the amino acid substitutes have protective properties. The protective amino acid substitutes comprise of amino acids with similar charges, sizes, and/or hydrophobic characteristics to the amino acids (one or more) they replace in the peptide. The aforementioned amino acid can be D-isomer or L-isomer amino acid residue, also can be natural or non-naturally occurring amino acid residue.

The antiviral peptide comprising the amino acid sequence of SEQ ID NO:1 can be jointly administered with other antiviral agents to achieve better therapeutic outcome, such as synergistic action or diminished side effect. These agents include those selected from nucleoside reverse transcriptase inhibitors, non-nucleoside reverse transcriptase inhibitors, protease inhibitors, fusion inhibitors, integrase inhibitors, among others. These agents are generally selected from the group consisting of 3TC (Lamivudine), AZT (Zidovudine), FTC (Emtricitabine), ddI (Didanosine), ddC (zalcitabine), ABC (abacavir), TDF (tenofovir), D4T (Stavudine), NVP (Nevirapine), DLV (Delavirdine), TMC-125 (etravirine), EFV (Efavirenz, Sustiva), SQVM (Saquinavir mesylate), RTV (Ritonavir), IDV (Indinavir), SQV (Saquinavir), NFV (Nelfinavir), APV (Amprenavir), Atazanavir (ATV), Fosamprenavir (FPV), LPV (Lopinavir, kaletra), TPV (Tipranavir), DRV (Darunavir), ENF (enfuvirtide, T20), MVC (Maraviroc), and MK-05 18 (Raltegravir) and mixtures thereof.

Therefore, the present invention also provides a method of treating viral infection caused by drug-resistant HIV comprising administering to the patient a therapeutically effective amount of peptides and other antiviral agents which can decrease viral load and increase CD4 cell count in HIV infected patient, a method of simultaneously, or sequentially administering the antiviral peptide and/or at least one antiviral agent to treat HIV infection. In one embodiment, the additional antiviral agent is selected from reverse transcriptase inhibitors, virus protease inhibitors, glycosidase inhibitors, viral mRNA capping inhibitors, amphotericin B, ester-bond binding molecules with anti-HIV activity, hydroxyurea, α-interferon, β-interferon, γ-interferon, and other anti-HIV agents.

The invention also contemplates treating HIV infection using gene therapy methods. Therefore, the invention includes administering to the patient subject a genetic material capable of producing inside the subject a therapeutically effective amount of the peptide of the invention. Such genetic materials can be DNA, RNA or other similar material that can reproduce a predetermined peptide sequence. The genetic material can be carried by a vector, a cell, a bacterium, a virus or another kind of carrier or host.

Certain expressions in the present invention are defined hereinafter:

As used herein, the term “anti-fusion” means to inhibit or suppress the fusion of two or more biological membranes that are of either cellular or viral structures, such as a cell membrane and a viral envelope. According to an embodiment of the present invention, the anti-fusion agent or anti-membrane fusion agent is a substance that inhibits the viral infection from cells by inhibiting the fusion of a cell membrane and a viral envelope or by inhibiting the fusion of cells.

The antiviral peptide in the present invention includes an amino acid sequence of SEQ ID NO:1 or conservatively modified variants thereof:

(SEQ ID NO: 1)

SWETWEREIENYTRQIYRILEESQEQQDRNERDLLE

As used herein, the single-letter codes represent amino acid residues in common sense in the art as follows: A=Alanine, R=Arginine, N=Asparagines, D=Aspartic acid, C=Cysteine, Q=Glutamine, E=Glutamic acid, G=Glycine, H=Histidine, I=lsoleucine, L=Leucine, K=Lysine, M=Methionine, F=Phenylalanine, P=Proline, S=Serine, T=Threonine, W=Tryptophan, Y=Tyrosine, V=Valine.

The antiviral peptide in the present invention can be manufactured by common techniques and methods known in the art. For instance, being synthesized on a certain solid or in solution, produced by recombinant DNA technology, or synthesized as several separated segments and then connected together.

As used herein, “conservatively modified” refers to an amino acid sequence having been structurally modified in a way that does not substantially influence the activity of the peptide, such as through amino acid substitution, terminal modification such as introduction of an acyl group, hydrophobic group or macromolecular carrier group and so on at one or two terminals. The conservatively modified peptide should retain at least 60% of the activity of the original peptide, preferably at least 80%, and more preferred, at least 90% of the biological activity of the original peptide.

As used herein, “viral infection” refers to a morbid state in which the virus invades a cell. When the virus enters a healthy cell, it utilizes the host reproduction mechanism to replicate itself, which finally kills the cell. After budding from the cell, those newly produced progeny viruses continue to infect other cells. Some viral genes can also integrate into host chromosome DNA in the form of provirus, and it is called as latent infection. The provirus reproduces itself with the replication of the host chromosome, and can bring the infected people into morbidity at any moment if activated by various factors inside or outside the body.

As used herein, “to treat HIV infection” refers to suppressing the replication and the spread of viruses, preventing the virus from settling inside the host, and improving or alleviating the symptoms caused by viral infection. The criteria for effective therapy includes the therapy's ability to lower the viral load, reconstruct immune system, lower mortality rate, and/or morbidity rate, etc.

As used herein, “derivatives” refers to any peptide that contains the entire sequence of the antiviral peptide, or a homolog, analog, or segment of the antiviral peptide, or to peptides that have substituted, inserted, and/or deleted one or more amino acids from the original sequence.

As used herein, a “therapeutic agent” refers to any molecule, compound, or drug conductive to the treatment of viral infection or virus-caused diseases, especially antiviral agents.

As used herein, “synergic effect” refers to a joint drug administration of two or more therapeutics that is more effective than the additive action of merely using any one of the two or more therapeutics to cure or to prevent viral infection. The synergic effect can increase the efficacy of antiviral drugs and prevent or reduce viral tolerance against any single therapeutic.

As used herein, an “effective amount” refers to an amount that is sufficient to suppress viral replication, or relieve the disorders associated with viral infections noted above. The “effective amount” is determined with reference to the recommended dosages of the antiviral parent compound. The selected dosage will vary depending on the activity of the selected compound, the route of administration, the severity of the condition being treated, and the condition and prior medical history of the patient being treated. However, it is within the skill of the art to start doses of the compound(s) at levels lower than required to achieve the desired therapeutic effect and to gradually increase the dosage until the desired effect is achieved. It will be understood, however, that the specific dose level for any particular patient will depend on a variety of factors, including the body weight, general health, diet, time, and route of administration and combination with other drugs, and the severity of the disease being treated.

As used herein, “mutation” refers to a change in an amino acid sequence or in a corresponding nucleic acid sequence relative to a reference nucleic acid or polypeptide.

As used herein, a virus or a pathogen becomes “drug-resistant” or acquires “drug resistance” when at least one drug meant to neutralize it now has a reduced effect. For example, this often refers to, but not necessarily always, in the context of HIV virus, the ability of the virus to mutate and reproduce itself in the presence of one or more antiretroviral agents. Drug resistance can be measured by a higher half maximal (50%) inhibitory concentration (IC50), which shows a pathogen's reduced sensitivity to a therapeutic agent.

### Administration Dose

In the treatment of acute viral infection in mammals such as human, antiviral peptide or its derivatives should be administered in effective amount sufficient to suppress viral replication. This effective dose can be determined by methods generally known to one of skill in the art, including setting parameters such as biological half-life period, bioavailability and toxicity, etc.

The dose of the drug depends upon the plasma concentration of the antiviral peptide which is to relieve the symptoms or to extend survival time of the patient. It can be determined through cell culture assay or animal studies according to standard pharmaceutical procedure.

It is more precise to get data from human trials. Viral replication, especially HIV concentration, can be suppressed by an effective amount of antiviral peptide in plasma, by administration the effective amount of pharmaceutically acceptable antiviral peptide every time, such as administration of 1-100 mg antiviral peptide.

In joint administration with antiviral peptide or its derivatives, the effective dosage of the therapeutic agents (such as antiviral agents) depend on the recommended doses for those agents, which are established and well-known to one skill in the art. The preferred dose for joint administration is, in one embodiment, about 10-50% lower than the recommended dose for separate administration in literature. Medical professionals should pay attention to the dose when toxic reactions occur. The doctor should know how and when to suspend or terminate the administration and to regulate the dose to a lower level when marrow function inadequacy, liver and/or kidney function inadequacy, or serious drug interaction occurs. In contrast, if the anticipated clinical therapeutic efficacy is not achieved, the doctor should also know how and when to enhance the dose according to accepted clinical practice.

### Dose Intervals

Dosage intervals to prevent or treat HIV infection depend on the speed of absorption, deactivation, excretion of the drug, and other factors well-known to one skill in the art. It should be noted that dose should be adjusted according to relief of the disease. Specific regimen should be individualizing administered and supervised, and the administration should adjust according to duration of treatment according to professionals. Dosage provided in the present invention is taken as an example, but not a limit to the range or practice of the pharmaceutical composition. The active component can be administered once, or subdivided into smaller doses according to diverse intervals to maintain the effective amount of active agents in human.

### Pharmaceutical Formulation and Administration Route

The best methods of giving the present antiviral peptide include (but are not limited to): injection (such as intravenous, intraperitoneal, intramuscular, and hypodermal injection, etc.); epithelium or mucosa absorption, such as actinal mucosa, rectal, vaginal epithelium, pharynx nasalis mucosa, enteric mucosa, etc; per os; transdermal or other pharmacologically feasible administration routes. In order to treat viral infection, patients are administrated directly with the antiviral peptide or drug composition containing the antiviral peptide to obtain the dose amount for treating viral infection. The preparation and administration technology for this application is well known to one of skill in the art.

The present invention also includes the use of pharmacologically acceptable carrier to prepare a proper dosage formulation of the antiviral peptide and/or pharmaceutical composition for different administration route.

Injection formulation is a sterilized or aseptic pharmaceutical formulation used for administration, divided into four types: solution, suspension, emulsion, and aseptic powder (powder injection). It is one of the most popular formulations, having many advantages—quick action, reliable, fit for drugs not suitable for oral administration, fit for patient unable to take medicine orally, may target local area. However, there are some drawbacks—inconvenience, pain during injection, and complicated manufacture process. Administration route depends on medical need, including intravenous injection, intraspinal injection, intramuscular injection, subcutaneous injection and intradermal injection.

Besides the active ingredient, supplemental substances are added into the injection to improve the efficacy, safety and stability of the injection during preparation. Commonly used supplemental substances are solubilizing agent, buffer, suspending agent, stabilizer, chelator, oxidation inhibitor, bacteriostatic, local anesthesia agent, isoosmotic adjusting agent, loading agent, protectant et al., for example, acetate, phosphate, carbonate, glycine, mannitol, lactose and human serum albumin. The preparation of drug can be performed by methods generally known to one of skill in the art—antiviral peptide can be prepared into aseptic aqueous solution without pyretogen—the solution can be distilled water, preferably saline water, phosphate buffered saline or 5% glucose solution. Selectively adjust the pH value to be physiologically acceptable, such as to subacidity or physiological pH value. After sterilization or aseptic filtration, transfer the solution to a unit container, e.g. a vial, and then freeze dry into a convenient dosage unit for use. Directions on how to use the drug should be noted on the inner/outer package of the dosage form, such as the weight or content of the active ingredient and administration interval. The antiviral peptides of the present invention can also be prepared as a solution fit for oral administration by use of a pharmacologically acceptable carrier well-known in the art. Appropriate carriers are necessary for preparing the peptides or combinations into tablets, pills, capsules, liquid, gel, syrups, slurry, suspensions, and other dosage formulations.

The antiviral peptides of the present invention can be administered by routes including actinal, rectal, dialysis membrane, or enteric administration; extragastrointestinal administration including intramuscular, hypodermal, intramedullary, introthecal, directly intraventricular, intravenous, intraperitoneal, intranasal, or intraocular injection; percutaneous, local, vaginal administration, etc. The dosage formulations include (but not limited to) tables, pastille, powders, suspensions, suppositories, solution, capsules, frost, plasters, and micro-motors.

The oral dosage formulation of the antiviral drug compositions of the present invention can be ground together with solid excipients into a well-distributed mixture and then processed into granules that are further processed into tablets or the kernel of sugar-coated tablets; if necessary, proper adjuvant can be added to the mixture. Proper excipients and fillers can be sugar, such as lactose, saccharose, mannitol, or sorbicolan; fibrin products, such as cornstarch, wheaten starch, rice starch, potato starch, glutin, tragacanth, methyl cellulose, hydroxypropylmethyl cellulose, sodium carboxymethyl cellulose, and/or polyvinylpyrrolidone. If necessary, disintegrants, such as cross-linked polyvinylpyrrolidone, agar, alginic acid, or its salt-like alginate sodium. Proper coat should be provided to the kernel of sugar-coated tablets. The coat can be made from concentrated sugar solution containing Arabic gum, talcum, polyvinylpyrrolidone, carbopol gel, polyethylene glycol, titanium oxide, cellulose nitrate, and proper organic solvent or solvent combination.. Different combinations of coloring matter or edible pigment can be added to the tablets or coat of sugar-coated tablets to discriminate or designate the active compound.

The drug composition for oral administration includes the stuffing-type capsule and the sealed soft capsule made of glutin and a plasticizer such as glycerin or sorbic acid. The stuffing-type capsule contains a filler, such as lactose, an adhesive, such as starch, and/or a lubricant, such as talcum or stearate. In addition, a stabilizer can also be used to stabilize the active components. In the soft capsule, the active compound can be dissolved or suspended in some proper liquid, such as fatty oil, liquid olefin, or liquid-like polyethylene glycol. Besides, a stabilizer can also be added. All the dosage formulations for oral administration should be convenient for patients. In the case of actinal administration, the above-mentioned combination can be prepared into the convenient dosage formulations of troche.

In the case of inhalation administration, the antiviral peptides or compositions of the present invention can be readily released in the form of aerosol by use of high-pressure package or atomizer, or by use of some proper propellant such as dichlorodifluoromethane, trichlorofluoromethane, dichlorotetrafluoroethane, carbon dioxide or other proper gases. In the case of high-pressure aerosol, the dosage unit can be defined by the quantity of measured release with one valve. The glutin capsule and cartridge used as insufflator or exsufflator can be produced as a mixture containing the antiviral peptides and a proper pulverous substrate (such as lactose or starch).

The antiviral peptides or compositions of the present invention can be prepared into a dosage formulation for extragastrointestinal administration. For example, they can be prepared into a formulation for injections that include cluster-drug injection or continuous intravenous infusion. The preparation for injection can be packed in the form of unit dosage. For example, it can be packed into ampoules. Preparations in large dosage can also be packed in the form of unit dosage, such as ampoule or large-dosage container, and added with preservative. The combinations of the present invention can take the form of suspension, solution or emulsion with oil or water as its medium, and can contain some additives, such as a suspending agent, stabilizer, and/or dispersant.

The drug compositions for extragastrointestinal administration can be in a water solution of the active substance, namely the water-dissolved form. The suspension of the active substance can also be produced as a proper oil-like suspension injection. The proper oleophilic solvent or vector includes fatty oil such as gingeli oil, or synthesized fatty acid ester such as ethyl oleate or triglyceride, or liposome. Water-like suspension for injection can contain substance that increases the suspension viscosity, such as sodium carboxymethyl cellulose, sorbic alcohol, and glucosan. The above-mentioned suspension can also contain selectively a proper stabilizer or substance that increases the compound solubility in order to prepare a high-concentration solution. The active component of the pulverous injection can be dissolved in some proper solvent, such as sterile water for injection that is in the absence of pyretogen, before administration.

The antiviral peptides or compositions of the present invention can also be prepared into rectal dosage formulations such as suppositories or retained enemas. They can be prepared with frequent substrate such as cacao butter or other glyceryl esters.

Apart from the dosage formulations that have been described, the antiviral peptides or drug compositions can also be prepared as long-acting dosage formulations that can be administered by hypodermal or intramuscular planting or intramuscular injection. Therefore, the peptides and its derivatives or drug combinations can be prepared with proper polymers, hydrophobes (oil emulsion, for example), ion exchange chromatography, or hardly soluble derivatives, such as hardly soluble salt.

The drug carriers for hydrophobic peptides of the present invention are a co-dissolved system of organic polymers and aqueous phase that blends with water and contains benzyl alcohol and non-polar surfactant. This co-dissolved system can be a VPD co-dissolved system. VPD is a solution containing 3% (W/V) benzyl alcohol, 8% (W/V) non-polar surfactant multiethoxyaether and 65% (W/V) polyethylene glycol 300 in absolute alcohol, while a VPD co-dissolved system (VPD: 5W) is prepared with VPD diluted in water by 1:1 and 5% glucose. This kind of co-dissolved system can dissolve hydrophobes better while it will produce low toxicity in systemic administration. As long as its solubility and toxicity are not changed, the proportions of the co-dissolved system can be altered greatly. In addition, the components of the co-dissolved carrier can also be changed. For example, other non-polar surfactant with low toxicity can be used to substitute for multi-ethoxyaether; the proportion of polyethylene glycol can also be changed; other biologically-blending polymers, such as polyvinylpyrrolidone, can be used to substitute for polyethylene; other sugar or polyose can be used to substitute for glucose.

The antiviral peptide composition can also include proper carrier-like excipients in solid or gel phase. These carriers or excipients include (but not limited to) calcium carbonate, calcium phosphate, various sugar, starch, cellulose derivatives, gelatin, or polymers, such as polyethylene glycol.

The pharmaceutical coinpositions of the present invention also include the combination of effective amount of active components to obtain the therapeutic purpose. The method of determining effective dose is well known to one of skill in the art.

### Joint Administration

The present invention also provides a joint administration of antiviral peptide with other agents, such as other antiviral agents, in the treatment of viral infection, especially HIV infection. These agents may or may not have the same sites or mechanisms of action as viral fusion inhibitors. As a result, cooperative or synergistic effects may result from joint drug administration.

The joint drug administration can prevent synplasm formation and HIV replication, and thus suppressing the reproduction of HIV in the patients. The joint administration of the present invention can also be used to alleviate or cure the diseases associated with HIV infection. For example, antiviral peptide can be administered jointly with antifunal agents, antibiotics, or other antiviral agents to suppress HBV, EBV, CMV infection and other accidental infection (including TB).

The present invention provides an improved antiviral therapy for the treatment of broad viral (including HIV) infection. In addition, the present invention provides a method of joint drug administration aimed at boosting the therapeutic effect, including the use of antiviral peptide, at least a different medicine, and/or a pharmacologically acceptable vector. The combination therapy can prevent the virus from building up a tolerance against each therapeutic alone, and at the same time reduce drug toxicity and enhance the therapeutic index.

According to an embodiment of the present invention, antiviral peptide can be administered alone or with other antiviral agents in all the following, including (but not limited to): simultaneous administration, sequential administration, periodic administration, and periodic therapy (for example, administration of an antiviral compound, then a second antiviral compound within a certain period of time, repeating such administration sequence (namely the period) to reduce possible drug resistance of the antiviral therapy).

The present invention also provides a new therapeutic method which can reduce the effective dose and toxicity of other antiviral therapeutic agents. Furthermore, joint administration of drugs can inhibit viral infection of host cells through different mechanisms, which not only increase the antiviral efficacy but also prevent the viruses from develop resistance against any therapy alone. The probability of successful treatment is therefore increased. And at the same time reduce drug toxicity and enhance the therapeutic index.

The agents used jointly with antiviral peptide include any drugs which are known or under experiment. According to a preferred embodiment of the present invention, the antiviral peptide or its derivatives are administered together with another agent with a different viral suppression mechanism. These agents include (but not limited to): antiviral agents, such as the cytokines rIFNα, rIFNβ, and rIFNγ; reverse transcriptase inhibitors, such as 3TC,AZT, FTC, ddI, ddC, d4T, ABC, NVP, DLV, EFV, Etravirine, Loviride and other dideoxyribonucleosides or fluorodideoxyribonucleoside; protease inhibitors, such as RTV, IDV, NFV, SQV, APV, ATV, FPV, LPV, TPV and DRV; integrase inhibitor MK-0518; hydroxyurea; viral mRNA capping inhibitors, such as viral ribovirin; amphotericin B; ester bond binding molecule castanospermine with anti-HIV activity; glycoprotein processing inhibitor; glycosidase inhibitors SC-48334 and MDL-28574; virus absorbent; CD4 receptor blocker; chemokine co-receptor inhibitor; neutralizing antibody; and other fusion and attachment inhibitors, such as Maraviroc, Enfuvirtide.

The peptide of the present invention can be administered jointly with retrovirus inhibitors, including (but not limited to) nucleoside derivatives. The nucleoside derivatives are improved derivatives of purine nucleosides and pyrimidine nucleosides. Their acting mechanism is to prevent RNA and DNA from being synthesized. The nucleoside derivatives, in the absence of any 3′ substituent that can be bound to other nucleosides, can suppress the synthesis of cDNA catalyzed by reverse transcriptase and thereby terminate the viral DNA replication. This is why they become anti-HIV therapeutic agents. For example, AZT and ddT, both of them can suppress HIV-1 replication in vivo and in vitro, had been approved as remedies for HIV infection and AIDS. However, use of these kinds of drugs alone for treatnent can lead to mass propagation of drug resistant HIV strains in addition to many side effects.

The nucleoside derivatives include (but not limited to): 2′,3′-dideoxyadenosine (ddA); 2′,3′-diseoxyguanosine (ddG); 2′,3′-dideoxyinosine (ddI); 2′,3′-dideoxycytidine (ddC); 2′,3′-dideoxythymidine (ddT); 2′,3′-dideoxy-dideoxythymidine (d4T) and 3′-azide2′,3′-dideoxycytidine (AZT). According to an embodiment of the present invention, the nucleoside derivatives are halonucleoside, preferably 2′,3′-dideoxy-2′-fluoronuceotides, including (but not limited to): 2′,3′-dideoxy-2′-fluoroadenosine; 2′,3′-dideoxy-2′-fluoroinosine; 2′,3′-dideoxy-2′-fluorothymidine; 2′,3′-dideoxy-2′-fluorocytidine; and 2′,3′-dideoxy-2′,3′-didehydro-2′-fluoronuceotides, including (but not limited to): 2′,3′-dideoxy-2′,3′-didehydro-2′fluorothymidine (F-d4T). More preferably, the nucleoside derivatives are 2′,3′-dideoxy-2′-fluoronuceotides wherein the fluorine bond is in the P conformation, including (but not limited to): 2′,3′-dideoxy-2β-fluoroadenosine (F-ddA), 2′,3′-dideoxy-2′β-fluoroinosine (F-ddI), and 2′,3′-dideoxy-2′β-fluorocytidine (F-ddC). Joint drug administration can reduce the dosage of nucleoside derivatives, and thereby reducing its toxicity as well as the chance of inducing drug-resistance in the surviving virus, while maintaining their antiviral potency.

Antiviral peptide can also be administered jointly with inhibitors of urdine phosphorylating enzyme, including (but not limited to) acyclouridine compounds, including benzylacyclouridine (BAU); benzoxybenzylacyclouridine (BBAU); amethobenzylacyclouridine (AMBAU); amethobenzoxybenzylacyclouridine (AMB-BAU); hydroxymethylbenzylacyclouridine (HMBAU); and hydroxymethylbenzoxybenzylacyclouridine (HMBBAU).

The antiviral peptide of present invention can also be administered jointly with cytokines or cytokine inhibitors, including (but not limited to): rIFNα, rIFNβ, and rIFNγ; TNFα inhibitors, MNX-160, human rIFNα, human rIFNβ and human rIFNγ. A more preferred joint drug administration includes the peptide.of the present invention and p interferon in effective dose.

Protease inhibitors prevent the virus from maturing mainly during the viral assembly period or after the assembly period (namely during the viral budding). Protease inhibitors show an antiviral activity both in vivo and in vitro. After administration of protease inhibitors, the HIV viral load in AIDS patient exhibits an exponential decline and their CD4 lymphocytes number rise (Deeks, et al., 1997, JAMA 277:145-53). Joint administration of viral protease inhibitors with fusion inhibitor can produce a synergic effect and achieve satisfactory clinical results. The present invention provides a method for treating HIV infection, which is a joint drug administration using the antiviral peptide in effective dose together with a protease inhibitor in effective dose, the latter including (but not limited to): Indinavir, Saquinavir, Ritonavir, Nelfinavir, Amprenavir, Atazanavir, Fosamprenavir, Tipranavir, Darunavir.

In addition, antiviral peptide in the present invention can be administered jointly with amphotericin B. Amphotericin B is a polyene antifungal antibiotic that can bind irreversibly with sterol. Amphotericin B and its formate have an inhibititory effect against many lipid enveloped viruses including HIV. Amphotericin B has a serious toxicity towards human body while its formate has a much lower toxicity. Thus, Amphotericin B or its formate can be administered jointly with antiviral peptide, and produce an anti-HIV synergic effect, which allows clinical doctors to use Amphotericin B or its formate in lower doses without losing its antiviral activities.

The antiviral peptide of the present invention can also be administered jointly with the glycoprotein processing inhibitor castanospermine, which is a vegetable alkaloid capable of inhibiting glycol protein processing. HIV envelope contains two large glycoproteins gp120 and gp41. The glycosylation of proteins plays an important role in the interactions between gp120 and CD4. The progeny virus synthesized in the presence of castanospermine has a weaker infectivity than the parental virus. The joint administration of antiviral peptide or its derivatives with castanospermine can produce a synergic effect.

The therapeutic effect of the joint administration of antiviral peptide with the above-mentioned antiviral therapeutics can be evaluated by generally used methods in the present field. For example, the joint effect of antiviral peptide and AZT can be tested through a variety of in vitro experiments including: inhibiting HIV toxicity against cells, inhibiting the formation of synplasm, inhibiting the activity of reverse transcriptase, or inhibiting viral ability for RNA or protein synthesis, etc.

According to an embodiment of the present invention, therapeutic agents that can be used jointly with antiviral peptide include (but not limited to): 2-deoxy-D-glucose (2dGlc), deoxynojirimycinacycloguanosine, virazole, rifadin, adamantanamine, rifabutine, ganciclover (DHPG), famciclove, buciclover (DHBG), fluoroiodoaracytosine, iodoxuridine, trifluorothymidine, ara-A, ara-AMP, bromovinyldeoxyuridine, BV-arau, 1-b-D-glycoarabinofuranoside-E-5-[2-bromovinyl]uracil, adamantethylamine, hydroxyurea, phenylacetic heptanedione, diarylamidine, (S)-(p-nitrobenzyl)-6-thioinosine and phosphonoformate. The present invention provides a drug combination of antiviral peptide with any other above-mentioned compounds.

The invention will now be described in greater detail by reference to the following non-limiting examples.

### Example

### Example 1

### Circular Dichroism (CD) Spectroscopy

Circular dichroism (CD) spectroscopy was performed following the method previously described in He, Y., et al. J.BIOL.CHEM. 2007; 282: 25631-25639. An N-peptide (N36) was incubated with equal molar concentration of a C-peptide (C34 or T20) or FS-01 at 37° C. for 30 min. N36 and C34, as described previously, are protease-resistant N- and C-peptides from gp41, respectively, which have been found to form the stable fusogenic core in gp41 of the HIV virus. The final peptide concentration was 10 μM in 50 mM sodium phosphate and 150 mM NaCl, pH 7.2. The N-, C-peptides as well as FS-01 by itself was also tested. CD spectra of these individual peptides and peptide mixtures were acquired on Jasco spectropolarimeter (Model J-715, Jasco Inc., Japan) at room temperature using a 5.0-nm band with, 0.1-nm resolution, 0.1-cm path length, 4.0-s response time, and a 50-nm/min scanning speed. The spectra were corrected by subtraction of a blank corresponding to the solvent. The a-helical content was calculated from the CD signal by dividing the mean residue ellipticity at 222 nm by the value expected for 100% helix formation (−33,000 degrees cm² dmol⁻¹) according to existing literature (Shu, W., et al. BIOCHEMISTRY 2000; 39: 1634-1642; Chen, Y. H., et al. BIOCHEMISTRY 1974; 13: 3350-3359). Thermal denaturation was monitored at 222 nm by applying a thennal gradient of 2° C./min in the range of 4-98° C. To determine the reversibility, the peptide mixtures were then cooled to 4° C. and kept in the CD chamber at 4° C. for 30 min, followed by monitoring of thermal denaturation as described above. The melting curve was smoothened, and the midpoint of the thermal unfolding transition (Tₘ) values was calculated using Jasco software utilities.

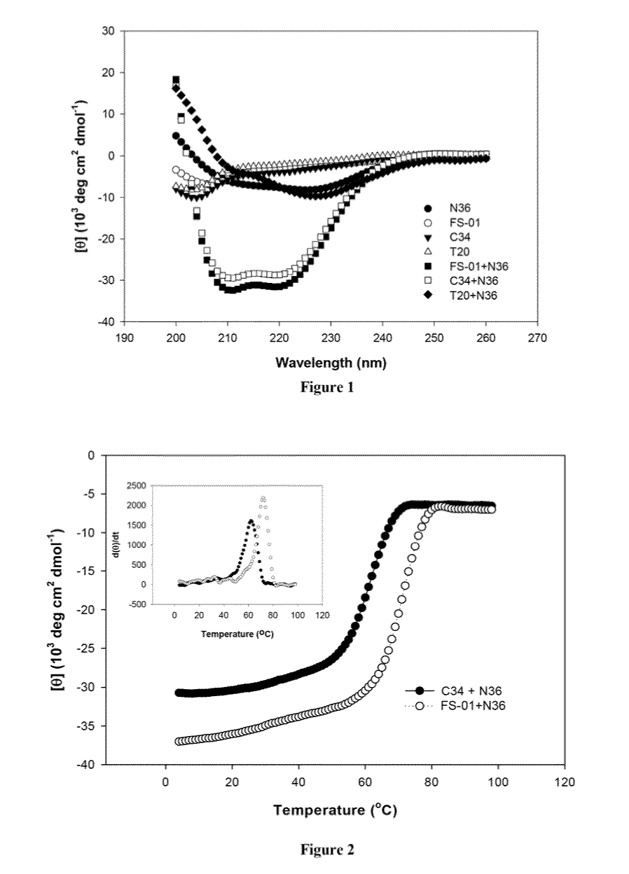

Referring to FIG. 1, the CD spectra of FS-01, C34, T20 and N36 did not show a minimum at 222 nm , suggesting that these peptides alone have minimal α-helical conformation. The CD spectrum for FS-01 is similar to that of C34 and T20 and is typical of peptide with a random coil conformation (Kliger, Y. and Shai, Y. J.MOL.BIOL. 2000, 295:163-168). In contrast, the CD spectra of FS-01 in the presence of N36 displays a double minimum at 208 and 222 nm, suggesting the interaction between FS-01 and N36 induces the formation of secondary α-helical structure. Similarly, mixing peptides C34 and N36 also led to the formation of α-helical complexes manifested by an almost overlapping CD profile (line of white square) with that of FS-01 combined with N36 (line of black square). Quantization of the CD data indicated a helical content of 93% for FS-01 and 85% for C34 peptide, both in the presence of N36. T20, on the other hand, did not form α-helical complexes with N36 peptide as demonstrated by the lack of minimum at 208 and 222 nm (line of black diamond). The result indicated that FS-01 binds to N36 in a comparable manner to C34 by forming a typical a-helix which is in great contrast to T20.

The CD thermal denaturation curves of FS-01/N36 complex in comparison with that of C34/N36 complex are diagramed in FIG. 2. The FS-01/N36 complex is found to have significantly higher level of α-helical content and is more heat-stable than that of C34/N36. The melting temperature (Tₘ) of FS-01/N36 complex is about 72° C. whereas that of C34/N36 is about 62° C. (FIG. 2, inset). The higher melting temperature by 10° C. suggests higher affinity between FS-01 and N36, and higher stability of FS-01/N36 complex compared to the C34/N36 complex.

### Example 2

### Native Polyacrylamide Gel Electrophoresis (N-PAGE)

Native polyacrylamide gel electrophoresis (N-PAGE) was carried out to determine the 6-HB formation between the N- and C-peptides using the described previously (Liu, S., et al. PEPTIDES 2003, 24:1303-1313). An N-peptide (N36) was mixed with a C-peptide (C34 or T20) or FS-01 at a final concentration of 40 μM and incubated at 37° C. for 30 min. The mixture was loaded onto a 10×1.0-cm precast 18% Tris-glycine gels (Invitrogen, Carlsbad, Calif.) at 25 μL/per well with an equal volume of Tris-glycine native sample buffer (Invitrogen). Gel electrophoresis was carried out with 125 V constant voltage at room temperature for 2 h. The gel was then stained with Coomassie blue and imaged with a FluorChem 8800 Imaging System (Alpha Innotech Corp., San Leandro, Calif.).

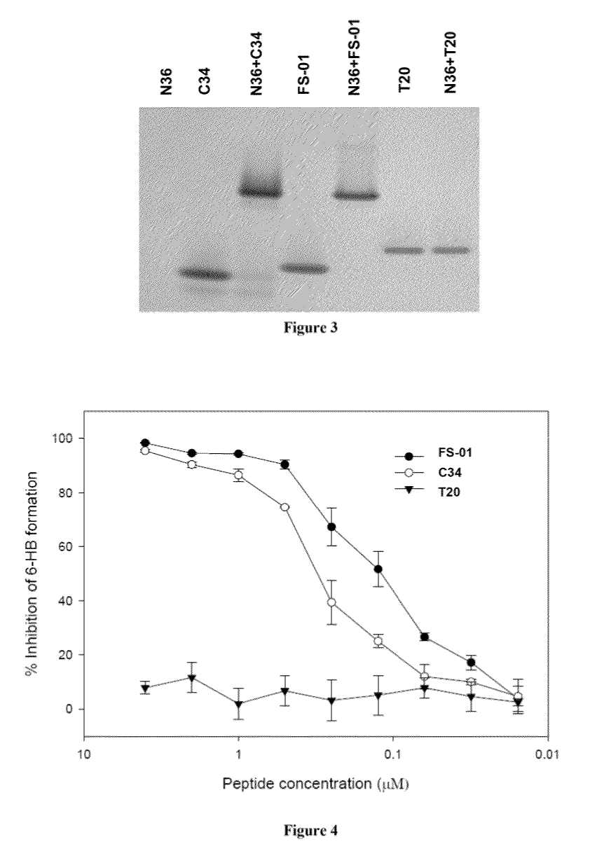

The result is shown in FIG. 3. N36 (lane 1, from left to right) exhibited no band because it carries net positive charges and might have migrated up and off the gel. C34 (lane 2), FS-01 (lane 4) and T20 (lane 6) each displayed a band in the lower part of the gel. Like C34, the result suggests that FS-01 was able to form 6-HB with N36 as reflected by the up-shift of a peptide band as compared to FS-0 1 or C34 alone. In contrast, no such changes were found for T20 after mixing with N36, suggesting that T20 and N36 did not form complexes similar to those of FS-01/36 or C34/N36.

### Example 3

### Inhibitory Activity of FS-01 on the 6-Helix Bundle Formation

Whether FS-01 can inhibit the formation of 6-HB between C34-biotin and N36 in solution was investigated by a founded antibody-based ELISA (Jiang, S., et al. J.VIROL.METHODS 1999; 80: 85-96). A 96-well polystyrene plate (Costar, Corning Inc., Corning, N.Y.) was coated with a 6-HB-specific monoclonal antibody NC-1 IgG (4 μg/mL in 0.1 M Tris, pH 8.8). A tested peptide (FS-01, C34 or T20) at graded concentrations was mixed with C34-biotin (0.25 μM) and incubated with N36 (0.25 μM) at room temperature for 30 min. The mixture was then added to the NC-1-coated plate, followed by incubation at room temperature for 30 min and washing with a washing buffer (PBS containing 0.1 % Tween 20) three times. Then streptavidin-labeled horseradish peroxidase (Invitrogen) and the substrate 3,3′,5,5′-tetramethylbenzidine (Sigma) were added sequentially. Absorbance at 450 nm (A450) was measured using an ELISA reader (Ultra 384, Tecan, Research Triangle Park, N.C.). The percent inhibition by the peptides and the IC₅₀ values were calculated as previously described (Jiang, S. B., et al J.EXP.MED. 1991; 174: 1557-1563).

FIG. 4 diagrams the result of the ELISA. Both FS-01 and C34 were able to disrupt the formation of 6-HB in a dosage-dependant manner whereas T20 failed to do so (FIG. 4). FS-01 demonstrated higher potency in disrupting the formation of 6-HB than C34, manifested by its IC₅₀ value of 0.12 μM compared to 0.31 μM of C34.

### Example 4

### HIV-1-1_{IIIB}-Mediated Cell-Cell Fusion and Infection

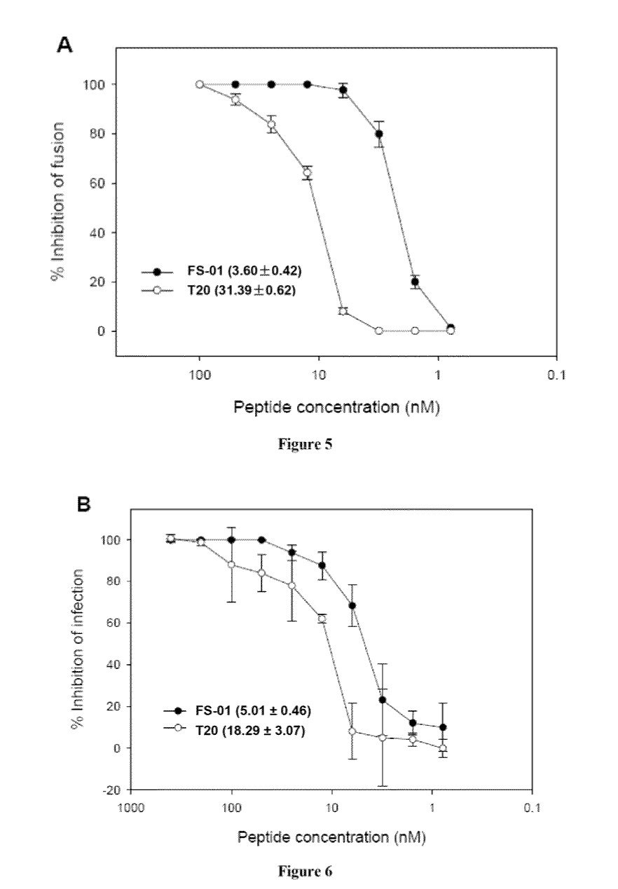

A dye transfer assay was used for detection of HIV-1 mediated cell-cell fusion as previously described in (Jiang, S., et al. Antimicrob.Agents Chemother. 2004; 48: 4349-4359. In brief, H9/HIV-1IIIB-infected cells were labeled with a fluorescent reagent, Calcein-AM (Molecular Probes, Inc., Eugene, OR) and then incubated with MT-2 cells (ratio =1:5) in 96-well plates at 37° C. for 2 h in the presence or absence of peptides. The fused and unfused calcein-labeled HIV-1-infected cells were counted under an inverted fluorescence microscope (Zeiss, Germany) with an eyepiece micrometer disc. The inhibition percent of cell-cell fusion and the IC50 values were calculated as described. FIG. 5 shows the result of the cell-cell fusion assay. FS-01 could inhibit the fusion with an IC50 of 3.60 nM. In comparison, T20 inhibited the fusion with an IC50 of 21.39 nM. Thus, FS-01 is about 6-fold more active than T20 in inhibiting membrane fusion. The inhibitory activity of FS-01 on HIV-1-mediated infection was conducted too. Consistently, FS-01 was also more potent to inhibit HIV-IIIIB-mediated infection of MT-2 cells than T20 (FIG. 6).

### Example 5

### Anti-HIV-1 Activity of FS-01

1) Inhibitory Activity against Primary HIV-1 Isolates

The inhibitory activity of FS-01 against primary HIV-1 isolates was determined as previously described in (Jiang, S., et al. ANTIMICROB. AGENTS CHEMOTHER. 2004; 48: 4349-4359. The peripheral blood mononuclear cells (PBMC) were isolated from the blood of healthy donors using a standard density gradient (Histopaque-1077, Sigma) centrifugation. The cells were plated in 75-cm plastic flasks and incubated at 37° C. for 2 h. The nonadherent cells were collected and resuspended at 5×10⁵/mL in RPMI 1640 medium containing 10% FBS, 5 μg of phytohemagglutinin (PHA)/mL, and 100 U of interleukin-2/mL, followed by incubation at 37° C. for 3 days. The PHA-stimulated cells were infected with the corresponding primary HIV-1 isolates at a multiplicity of infection (MOI) of 0.01 in the absence or presence of FS-01 at graded concentrations. Culture media were changed every 3 days. The supernatants were collected 7 days post infection and tested for p24 antigen by ELISA. The percent inhibition of p24 production and IC₅₀/IC₉₀ values were calculated. As shown in Table 1, FS-01 was highly active in inhibiting primary HIV-1 isolates with distinct genotypes and phenotypes (subtypes A to F), in particular against X4R5 viruses.

TABLE 1
Potent inhibition of SFT on primary HIV-1 isolates

| | FS-01 (Mean ± SD, nM) | | T20 (Mean ± SD, nM) | |
|---|---|---|---|---|
| Virus | IC₅₀ | IC₉₀ | IC₅₀ | IC₉₀ |
| 94UG103 (clade A, X4R5) | 3.94 ± 1.29 | 7.43 ± 2.16 | 83.84 ± 12.09 | 150.69 ± 8.80 |
| 92US657 (clade B, R5) | 27.78 ± 8.66 | 211.78 ± 83.96 | 41.45 ± 8.76 | 177.33 ± 15.95 |
| 93IN101 (clade C, R5) | 34.31 ± 3.21 | 90.64 ± 6.50 | 47.21 ± 4.73 | 129.69 ± 16.76 |
| 92UG001 (clade D, X4R5) | 11.92 ± 3.01 | 35.68 ± 6.59 | 185.10 ± 23.34 | 501.40 ± 70.78 |
| 92TH009 (clade E, R5) | 209.77 ± 9.91 | 618.65 ± 39.04 | 221.44 ± 58.31 | 724.35 ± 56.64 |
| 93BR020 (clade F, X4R5) | 12.46 ± 6.76 | 71.64 ± 28.54 | 73.68 ± 8.57 | 204.26 ± 28.01 |

2) Inhibitory Activity against T20-Resistant Virus Isolates and Laboratory-Adapted HIV-1 Strain (HIV-1_{IIIB})

Two T20-sensitive and five T20-resistant strains with well defined genetic mutations conferring the resistance were used in this experiment (Rimsky, L. T., et al. J.VIROL. 1998; 72: 986-993). Briefly, 1×10⁴ MT-2 cells were infected with HIV-1 isolates at 100 TCID₅₀ (50% tissue culture infective dose) in 200 μl RPMI 1640 medium containing 10% FBS in the presence or absence of FS-01 at graded concentrations overnight. Then the culture supernatants were removed and fresh media without FS-01 were added. On the fourth day post-infection, 100 μl of culture supernatants were collected from each well, mixed with equal volumes of 5% Triton X-100 and assayed for p24 antigen by ELISA. It is found that both FS-01 and T20 were active against T20-sensitive strains, but FS-01 was more potent than T20. More importantly, FS-01 showed strong inhibitory activities against all T20-resistant strains with IC₅₀ ranging from 2.68 to 47.78 nM while T20 failed to do so even with concentration at least 40- to 700-fold higher (Table 2). This provides strong evidence of FS-01 's efficacy against HIV strains that had developed resistance to existing therapeutics, in this case, a fusion inhibitor T20.

TABLE 2
Potent Inhibition of SFT on T20-resistant viruses

| | | IC₅₀ (Mean ± SD, nM) | |
|---|---|---|---|
| Virus | Phenotype* | T20 | FS-01 |
| NL4-3 (parental) | S | 22.04 ± 1.19 | 12.56 ± 1.18 |
| NL4-3 (N42S) | S | 26.95 ± 12.98 | 2.68 ± 0.87 |
| NL4-3 (V38A) | R | >2000 | 3.44 ± 0.23 |
| NL4-3 (V38A/N42D) | R | >2000 | 47.78 ± 4.70 |
| NL4-3 (V38A/N42T) | R | >2000 | 30.42 ± 4.36 |
| NL4-3 (V38E/N42S) | R | >2000 | 43.47 ± 3.36 |
| NL4-3 (N42T/N43K) | R | >2000 | 37.79 ± 15.55 |

*Sensitive (S) or resistant (R) to T20

### Example 6

### Peptide-Lipid Binding Activity of FS-01

Previous studies suggest that the CHR helices located in the outer layer of the 6-HB may interact with the lipid membranes and participate in the formation of fusion pore (Shu, W., et al. J. BIOL. CHEM. 2000; 275: 1839-1845). So, the POPC LUV liposome system was employing to investigate the binding activity of FS-01 to lipid membranes. Large unilamellar vesicles (LUVs) of 1-palmitoyl-2-oleoyl-sn-glycero-3-phosphocholine (POPC) liposome were prepared as previously described (Liu, S., et al. J.BIOL.CHEM. 2007; 282: 9612-9620). Briefly, 20 mg of POPC stock solution was dried under a stream of nitrogen and stored in vacuum overnight to completely remove trace amounts of organic solvent. The dried lipid film was suspended by vortexing in 2 mL PBS buffer. The resultant multilamellar vesicle suspension was freeze-thawed for 5 cycles and then successively extruded through two stacked polycarbonate filters (0.1 μm) 13 times using an Avanti miniextruder.

### 1) Isothermal Titration Calorimetry (ITC)

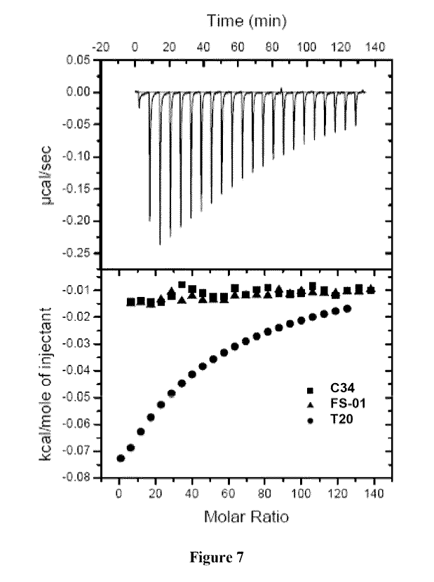

Binding of FS-01, C34 or T20 to POPC LUVs was measured using a high-sensitivity isothermal titration calorimetry (ITC) instrument (MicroCal, Northampton, Mass.) (Liu, S., et al. J.BIOL.CHEM. 2007; 282: 9612-9620). Solutions were degassed under vacuum prior to use. LUVs of POPC were injected into the chamber containing a peptide solution or buffer only (as a control). The heats generated in control experiments by injecting lipid vesicles into buffer without a peptide were subtracted from the heats produced in the corresponding peptide-lipid binding experiments. Data acquisition and analyses were performed using MicroCal's Origin software (Version 7.0). As shown in FIG. 7 (upper panel), large heat release was detected when POPC LUVs was added into the solution containing T20. The calculated binding constant of T20 to POPC LUVs was about 5×10⁴ M⁻¹. However, the bindings of FS-01 and C34 to POPC LUVs were too weak to calculate the binding constant (FIG. 7, lower panel).

### 2) Fluorescence Spectroscopy

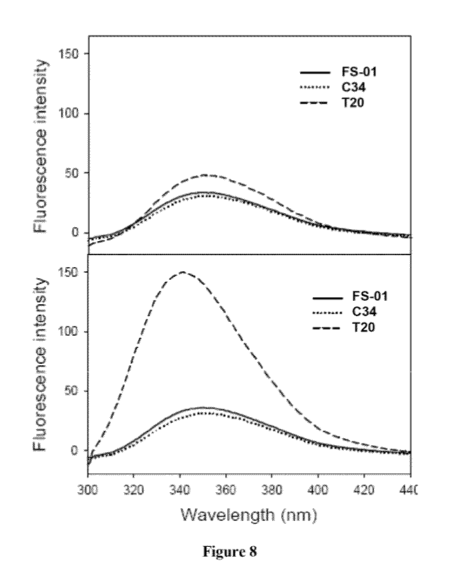

Because FS-01, C34 and T20 contain tryptophan residues, the tryptophan fluorescence emission spectra of the peptides may change if they interact with the lipid bilayers. Accordingly, fluorescence spectra of the peptides FS-01, C34 and T20 (10 μM) presented in PBS and POPC LUVs (2 mM), respectively, were obtained on a Hitachi fluorescence spectrophotometer, with an excitation wavelength of 295 nm, and emission was scanned from 300 to 450 nm at a scan rate of 10 nm/s. Spectra were baseline-corrected by subtracting blank spectra of the corresponding solutions without peptide. Significant blue shift of the fluorescence spectra was observed when T20 was presented in the POP LUVs (FIG. 8, lower panel), compared to its presence in PBS (FIG. 8, upper panel), whereas FS-01 and C34 did not show remarkable fluorescence spectra shift.

This result, along with the ITC result, suggests that unlike T20, both FS-01 and C34 may not interact with the target cell membrane.

Although the foregoing invention has been described in some detail by way of illustration and example for purposes of clarity and understanding, it will be apparent those skilled in the art in light of the teaching of this invention that certain changes or modifications may be made thereto without departing from the spirit and scope of the present invention as defined by the following claims.

We claim:

1. A method of treating drug-resistant HIV infection in a subject in need thereof, comprising administering to said subject at least a therapeutically effective amount of a peptide comprising the amino acid sequence of:
SWETWEREIENYTRQIYRILEESQEQQDRNERDLLE (SEQ ID NO:1) or a conservatively modified variant thereof.

2. The method of claim 1, wherein the peptide comprises the amino acid sequence of: X-SWETWEREIENYTRQIYRILEESQEQQDRNERDLLE-Z, wherein X is an amino group, acetyl group, a hydrophobic group, or a macromolecular carrier group; and wherein Z is a carboxyl group, amino group, a tert-butyloxycarbonyl group, a hydrophobic group, or a macromolecular carrier group.

3. The method of claim 2, wherein the hydrophobic group is selected from the group consisting of a carbobenzoxy, a dansyl, a tert-butyloxycarbonyl, and a 9-fluorenylmethyloxycarbonyl; and wherein the macromolecular carrier group is selected from the group consisting of a lipid-fatty acid chelate, a polyethylene glycol, and a carbohydrate.

4. The method of claim 2, wherein the peptide comprises the amino acid sequence of:
CH₃CO-SWETWEREIENYTRQIYRILEESQEQQDRNERDLLE-NH₂.

5. The method of claim 1, wherein the HIV is resistant to at least one reverse transcriptase inhibitor.

6. The method of claim 5, wherein the HIV is resistant to at least one nucleoside reverse transcriptase inhibitor.

7. The method of claim 5, wherein the HIV is resistant to at least one non-nucleoside reverse transcriptase inhibitor.

8. The method of claim 1, wherein the HIV is resistant to at least one protease inhibitor.

9. The method of claim 1, wherein the HIV is resistant to at least one fusion inhibitor.

10. The method of claim 9, wherein the fusion inhibitor is T20.

11. The method of claim 1, wherein the HIV is cross resistant to at least two members selected from the group consisting of a nucleoside reverse transcriptase inhibitor, a non-nucleoside reverse transcriptase inhibitor, a protease inhibitor and a fusion inhibitor.

12. The method of claim 1, further comprising administering to the subject a pharmaceutically acceptable excipient, carrier or diluent.

13. The method of claim 1, wherein the peptide is administered in a manner selected from the group consisting of intramuscular, intravenous, subcutaneous, oral, mucosal, rectal and percutaneous administration.

14. A method of treating drug-resistant HIV infection in a subject in need thereof, comprising administering to said subject at least:
(a) a therapeutically effective amount of a peptide comprising the amino acid sequence of:
SWETWEREIENYTRQIYRILEESQEQQDRNERDLLE (SEQ ID NO:1) or a conservatively modified variant thereof, and
(b) a second agent selected from the group consisting of reverse transcriptase inhibitors, protease inhibitors, integrase inhibitors, glycosidase inhibitors, viral mRNA capping inhibitors, amphotericin B, ester-bond binding molecules with anti-HIV activity, hydroxyurea, α-interferon, β-interferon, γ-interferon, and other anti-HIV agents.

15. The method of claim 14 wherein the reverse transcriptase inhibitor is at least one component selected from the group consisting of 3TC,AZT, FTC, ddI, ddC, d4T, NVP, DLV, EFV, Etravirine, PMEA, PMPA, and Loviride.

16. The method of claim 14 wherein the protease inhibitor is at least one component selected from the group consisting of RTV, IDV, NFV, SQV, APV, ATV, FPV, LPV, TPV and DRV.

17. The method of claim 14 wherein the integrase inhibitor is MK-0518.

18. The method of claim 14 wherein the glycosidase inhibitor is SC-48334 or MDL-28574 or both.

19. The method of claim 14 wherein the viral mRNA capping inhibitor is Ribovirin.

20. The method of claim 14, wherein the peptide is administered in a manner selected from the group consisting of intramuscular, intravenous, subcutaneous, oral, mucosal, rectal and percutaneous administration.

21. A method of treating drug-resistant HIV infection in a subject in need thereof, comprising administering to said subject at least a genetic material capable of producing inside the subject a therapeutically effective amount of a peptide comprising the amino acid sequence of:
SWETWEREIENYTRQIYRILEESQEQQDRNERDLLE (SEQ ID NO:1) or a conservatively modified variant thereof.


SEQUENCE LISTING (1 sequence)

SEQ ID NO: 1 - protein, 36 aa, homo sapiens
  HIV (1)..(36): HIV gp41
SWETWEREIENYTRQIYRILEESQEQQDRNERDLLE